(19) United States
(12) Patent Application Publication
(10) Pub. No.: US 2013/0274737 A1
(43) Pub. Date: Oct. 17, 2013

(54) RENAL NERVE MODULATION CATHETER DESIGN

(71) Applicant: BOSTON SCIENTIFIC SCIMED, INC., Maple Grove, MN (US)

(72) Inventors: HUISUN WANG, MAPLE GROVE, MN (US); DEREK C. SUTERMEISTER, HAM LAKE, MN (US); JAMES M. ANDERSON, FRIDLEY, MN (US)

(73) Assignee: BOSTON SCIENTIFIC SCIMED, INC., MAPLE GROVE, MN (US)

(21) Appl. No.: 13/864,115

(22) Filed: Apr. 16, 2013

Related U.S. Application Data
(60) Provisional application No. 61/624,913, filed on Apr. 16, 2012.

(51) Int. Cl. A61B 18/14

(52) U.S. Cl. CPC A61B 18/14
USPC 606/41

(57) ABSTRACT

System for nerve modulation and method for making and using the same are disclosed. An example system may include an elongate shaft having a proximal end region, a deflectable distal end region and a lumen extending to the deflectable distal end region. The deflectable distal end region may include an electrode surrounded by a permeable membrane. The permeable membrane may be fluidly connected to the lumen.

### CROSS-REFERENCE TO RELATED APPLICATIONS

This application claims priority under 35 U.S.C. §119 to U.S. Provisional Application Ser. No. 61/624,913, filed Apr. 16, 2012, the entirety of which is incorporated herein by reference.

### TECHNICAL FIELD

The present invention relates to methods and apparatuses for nerve modulation techniques such as ablation of nerve tissue or other destructive modulation technique through vessel walls and adjacent tissue.

### BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves, including renal nerves, run along the walls of or in close proximity to blood vessels and thus can be accessed via the blood vessels. In some instances, it may be desirable to ablate perivascular renal nerves using a radio frequency (RF) electrode. However, such a treatment may result in thermal injury to the vessel wall at the electrode and other undesirable side effects such as, but not limited to, blood damage, clotting and/or protein fouling of the electrode. Increased cooling in the region of the nerve ablation may reduce such undesirable side effects. It is therefore desirable to provide for alternative systems and methods for intravascular nerve modulation.

### SUMMARY

The disclosure is directed to several alternative designs, materials and methods of manufacturing medical device structures and assemblies for partially occluding a vessel and performing nerve ablation.

Accordingly, one illustrative embodiment is directed to a system for nerve modulation that includes an elongate catheter shaft having a lumen for delivering fluid to a distal end region. The distal end region may include an electrode surrounded by a permeable membrane. The distal end region may also include one or more spacers for keeping the electrode and the permeable membrane from contacting a vessel wall. The catheter may further include a deflection wire such as a pull wire for deflecting the distal end region in order to position the distal end region against a vessel wall. The permeable membrane may be, for example, a woven mesh.

The one or more spacers may be annular or helical and may include one or more gaps betweens segments of the spacers to allow for fluid flow past the spacer. The electrode may be a wire or coil within the lumen at the distal end region or may be a tubular member having a plurality of ports and providing structural support to the permeable membrane.

One illustrative embodiment is directed to a system for nerve modulation that includes a catheter having a distal end region with an electrode disposed in the distal end region and a plurality of bumpers and ports at the distal end region. The ports are fluidly connected to the lumen and the bumpers may be configured to keep the ports spaced from a vessel wall. The bumpers may be hollow and the ports may be disposed on the bumpers and connected through the bumpers to the lumen. The bumpers may comprise a plurality of annular rings, a helical member, or other suitable configuration and may include gaps between adjacent segments to allow for fluid flow.

One illustrative member is directed to a system for renal nerve modulation where the distal end region has a non-circular, cross-sectional shape that provides for a generally flat face. An electrode is disposed in the catheter lumen and at least one port is provided in the flat face. A deflection member may also be provided in the catheter lumen or the electrode may serve as the deflection member as well.

In addition to nerve modulation, the present apparatus and methods can be applied to modulation or ablation of other tissues in the body.

Some embodiments pertain to a method of performing an intravascular procedure, comprising the steps of providing a system as described herein, providing saline through the lumen and activating the electrodes to treat and/or ablate nerve tissue proximate the distal end region. Methods may also include the step of deflecting the distal end region to a position proximate a region of the vessel wall.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 is side view illustrating the distal portion of a renal nerve modulation system in situ.

FIG. 3 is a partial cross-sectional side view illustrating the distal portion of a renal nerve modulation system.

FIG. 4 is a cross-sectional side view illustrating the distal portion of a renal nerve modulation system.

FIG. 5 is a cross-sectional side view illustrating the distal portion of a renal nerve modulation system.

FIG. 6 is a partial cross-sectional side view illustrating the distal portion of a renal nerve modulation system.

FIG. 7 is a side view illustrating the distal end portion of a renal nerve modulation system.

FIG. 8 is a partial cross-sectional side view illustrating the distal portion of a renal nerve modulation system.

FIG. 9 is a partial cross-sectional side view illustrating the distal portion of a renal nerve modulation system.

FIG. 10 is a cross-sectional side view illustrating the distal portion of a renal nerve modulation system.

FIG. 11 is a cross-sectional side view illustrating the distal portion of a renal nerve modulation system.

FIG. 12 is a cross-sectional side view illustrating the distal portion of a renal nerve modulation system.

FIG. 13 is a side view illustrating the distal end portion of a renal nerve modulation system.

FIG. 14 is an isometric view illustrating the distal end portion of a renal nerve modulation system.

FIG. 15 is a cross-section end view illustrating the distal end portion of a renal nerve modulation system in situ.

FIG. 16 is a cross-section end view illustrating the distal end portion of a renal nerve modulation system in situ.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. In some instances, it may be desirable to ablate perivascular renal nerves with deep target tissue heating. However, as energy passes from an electrode to the desired treatment region the energy may heat the fluid (e.g. blood) and tissue as it passes. As more energy is used, higher temperatures in the desired treatment region may be achieved thus resulting in a deeper lesion. However, this may result in some negative side effects, such as, but not limited to thermal injury to the vessel wall, blood damage, clotting and/or protein fouling of the electrode. Positioning the electrode away from the vessel wall may provide some degree of passive cooling by allowing blood to flow past the electrode. However, it may be desirable to provide an increased level of cooling over the passive cooling generated by normal blood flow. In some instances, a partial occlusion catheter may be used to partially occlude an artery or vessel during nerve ablation. The partial occlusion catheter may reduce the cross-sectional area of the vessel available for blood flow which may increase the velocity of blood flow in a region proximate the desired treatment area while minimally affecting the volume of blood passing, if at all. The increased velocity of blood flow may increase the convective cooling of the blood and tissues surrounding the treatment area and reduce artery wall thermal injury, blood damage, and/or clotting. The increased velocity of blood flow may also reduce protein fouling of the electrode. The renal nerve modulation systems described herein may include other mechanisms to improve convective heat transfer, such as, but not limited to directing flow patterns with surfaces, flushing fluid from a guide catheter or other lumen, or infusing cool fluid.

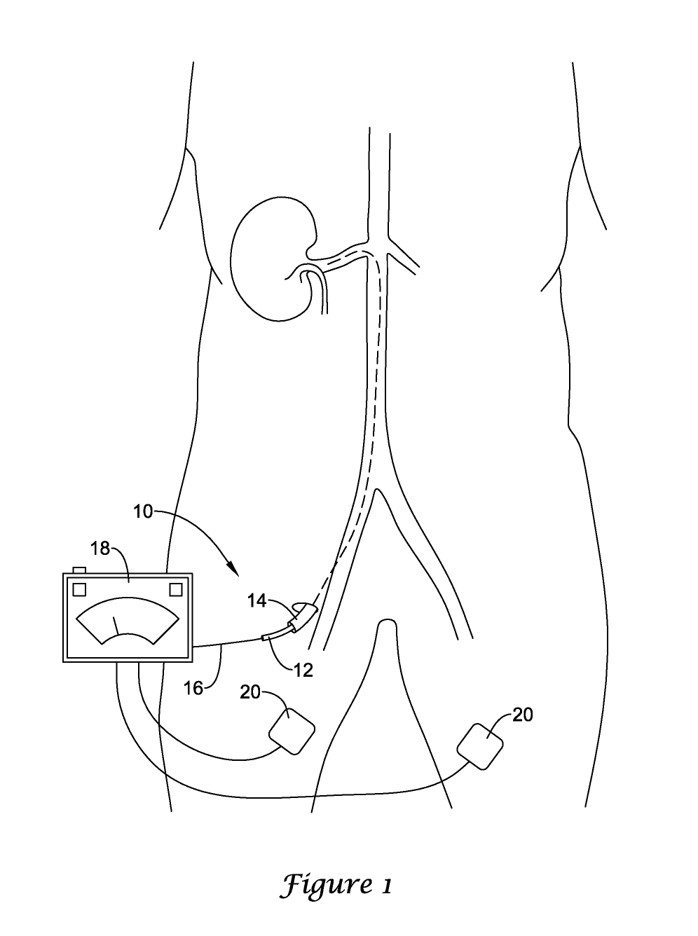

FIG. 1 is a schematic view of an illustrative renal nerve modulation system 10 in situ. System 10 includes a catheter 12 that includes a conductor 16 for providing power to an electrode (not illustrated) disposed within the catheter 12. The system 10 may include other elements such as a guide catheter 14. A proximal end of conductor 16 may be connected to a control and power element 18, which supplies the necessary electrical energy to activate the one or more electrodes in the distal end region of the catheter 12. In some instances, return electrode patches 20 may be supplied on the patient's back or at another convenient location on the patient's body to complete the circuit. The control and power element 18 may include monitoring elements to monitor parameters such as power, temperature, voltage, pulse size and/or shape and other suitable parameters as well as suitable controls for performing the desired procedure. In some instances, the power element 18 may control a radio frequency (RF) electrode. The electrode may be configured to operate at a frequency of approximately 460 kHz. It is contemplated that any desired frequency in the RF range may be used, for example, from 450-500 kHz. Lower or higher frequencies may be used, such as 10 kHz or 1000 kHz, in some cases, although the desired heating depth, catheter size, or electrical effects can limit the choice of frequency. However, it is contemplated that different types of energy outside the RF spectrum may be used as desired, for example, but not limited to ultrasound, microwave, and laser.

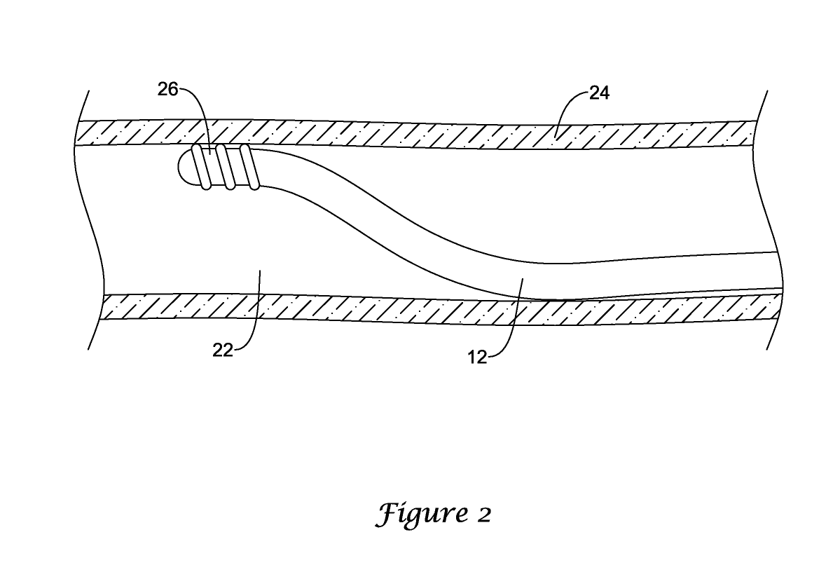

FIG. 2 is an illustrative embodiment of a distal end of a renal nerve modulation catheter 12 disposed within a body lumen 22 having a vessel wall 24. The catheter 12 may include an elongate shaft having a distal end region 26. The elongate shaft may extend proximally from the distal end region 26 to a proximal end configured to remain outside of a patient's body. The proximal end of the catheter 12 may include a hub attached thereto for connecting other treatment devices and/or providing a port for facilitating other treatments. The catheter 12 may further include one or more lumens extending therethrough. For example, the catheter 12 may include a guidewire lumen and/or one or more inflation lumens. The lumens may be configured in any way known in the art. For example, the guidewire lumen may extend the entire length of the catheter 12 such as in an over-the-wire catheter or may extend only along a distal portion of the catheter 12 such as in a single operator exchange (SOE) catheter. These examples are not intended to be limiting, but rather examples of some possible configurations. While not explicitly shown, the system 10 may further include temperature sensors/wire, an infusion lumen, radiopaque marker bands, fixed guidewire tip, a guidewire lumen, external sheath and/or other components to facilitate the use and advancement of the system 10 within the vasculature may be incorporated.

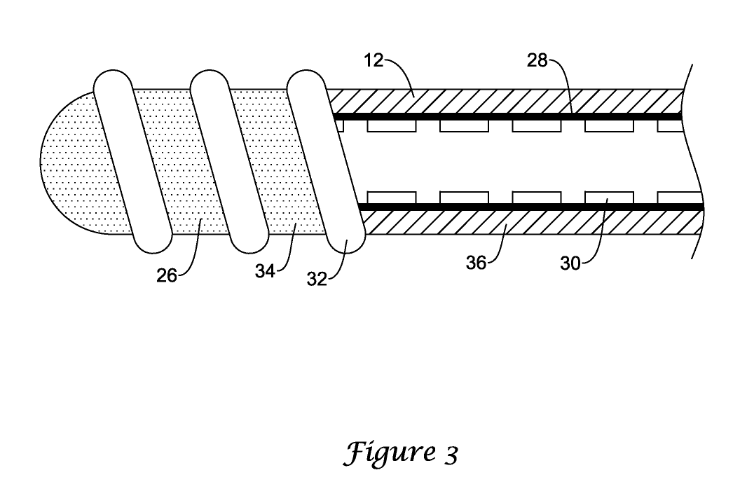

As shown in FIG. 3, the catheter 12 includes an electrode such as coil 30 disposed within a lumen of the catheter 12. One or more spacers 32 may surround a permeable material such as a polymeric woven mesh 34 or other suitable permeable material. The permeable material is fluidly connected to the catheter lumen such that fluid introduced into the lumen may exit through the interstices of the permeable material. In some embodiments, for example, the wall 36 of the catheter 12 terminates proximal the distal end region. In other embodiments, the wall 36 of the catheter includes one or more ports that allow fluid to pass through to the permeable material.

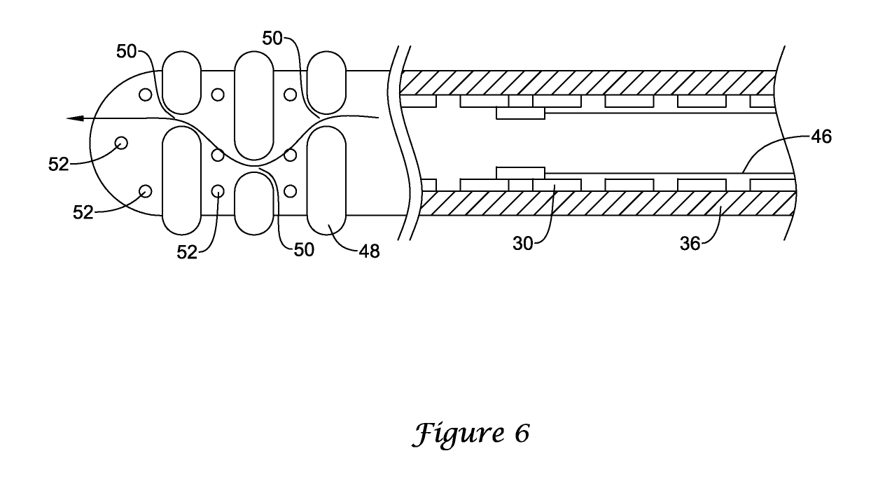

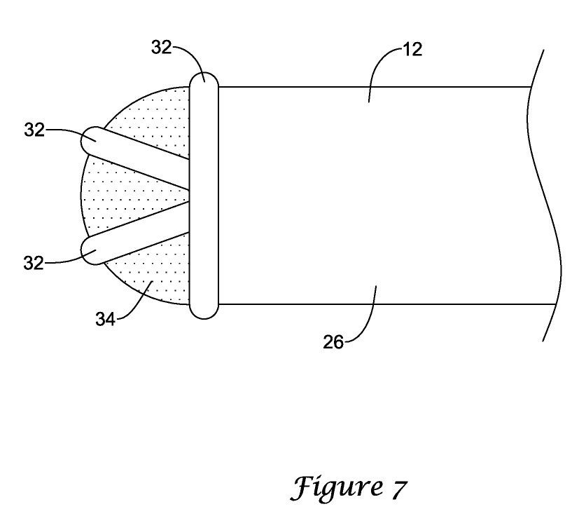

The spacers 32 may be helically shaped as shown in FIG. 2, may comprise annular rings (as shown, for example, in FIG. 6), may comprise distal features to keep the distal tip of the distal end region 26 from contacting the vessel wall (as shown, for example, in FIG. 7), may include gaps 50 between various segments of the spacers (as shown, for example, in FIG. 6) and may include other desired features. For example, although not explicitly shown in the figures, one contemplated embodiment includes spacers that have a generally helical configuration and also include gaps between the various segments of the helixes. Any spacer configuration that keeps the permeable material from the vessel wall may be suitable.

A catheter 12 may include a deflection member 28 such as a pull wire that can be actuated to move the catheter from a generally straight configuration to one in which the distal end region is proximate a vessel wall 24 as shown in FIG. 2. The distal end region 26 shown in FIG. 6 is illustrated as lying parallel to the vessel wall 24. In other embodiments, the distal end region 26 may be at an angle to the vessel wall 24.

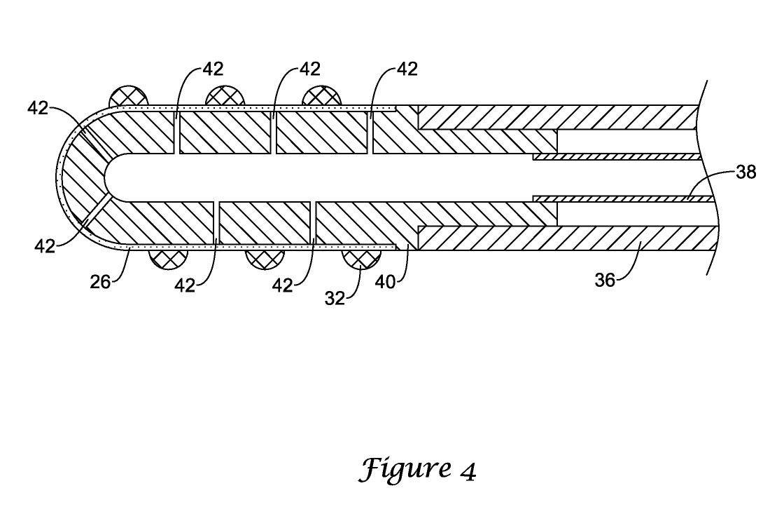

FIG. 4 illustrates a configuration where the wall 36 of the catheter terminates proximal to the distal end region of the catheter. The electrode in this configuration is a tube 40 having a plurality of ports 42 therethrough. The tube 40 may have a closed distal end as illustrated and the distal end may be rounded. The woven mesh 34 may partially or completely surround the tube 40 and a plurality of spacers 32 may be disposed in the distal end region 26 around the woven mesh 34. An inner tubular member 38 may be fluidly connected to the tube 40. This tubular member 38 may define the lumen that introduces saline into the tube 40 and may also comprise a conductive material to supply power to the tube 40. The catheter 12 of this embodiment may also include a deflection member (not illustrated) and other features discussed with respect to other embodiments.

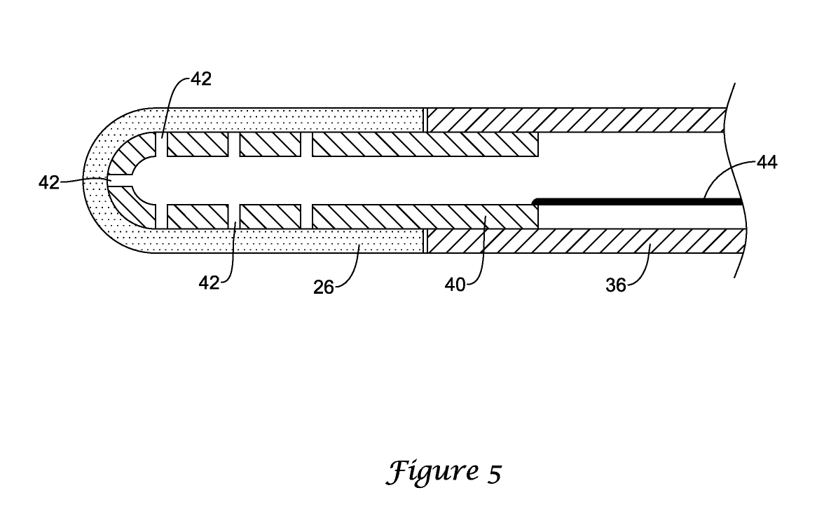

FIG. 5 illustrates a configuration similar to that of FIG. 4 where the electrode is a tube 40 having a plurality of ports therein. An element 44, which may be a conductor that supplies power to the tube 40, may also be included. In some variations of this embodiments, element 44 may also act as a pull wire. The distal end region may include a woven mesh 26 that fully surrounds the tube 40 electrode.

FIG. 6 illustrates an embodiment where the electrode is a coil 30 and power is supplied the coil by conductors 46. Conductors 46 may also act as deflection wires. In this embodiment spacers 48 are configured as annular ridges having gaps 50 to allow for fluid flow. The wall 36 of the catheter 12 extends to a closed distal end of distal end region and includes a plurality of ports 52 to allow for fluid flow from the catheter lumen.

FIG. 7 illustrates an embodiment where the spacer 32 curves around the distal tip of the catheter to prevent the distal tip of the catheter from touching the vessel wall. The catheter 12 includes other features previously discussed such as the woven mesh 34 and an electrode in a vessel lumen (not illustrated).

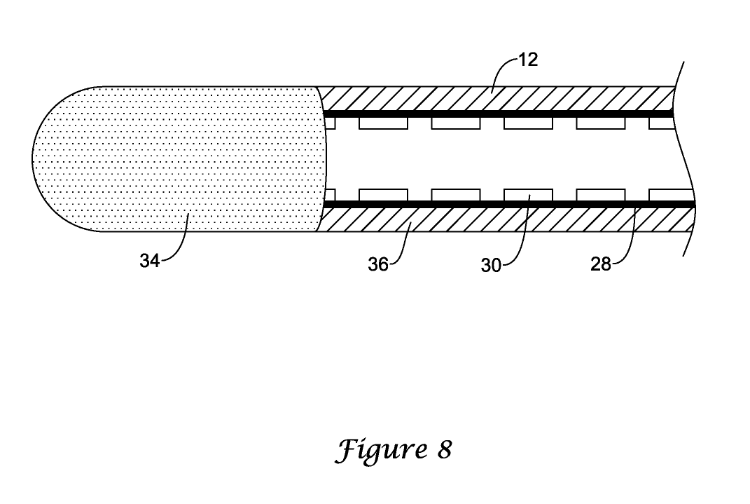

FIG. 8 illustrates an embodiment that is similar to that of FIG. 3 except as discussed herein. The embodiment lacks the spacers of the FIG. 3 embodiment. It will be appreciated that other internal configurations, such as that of FIG. 4 may be used in variations of this embodiment.

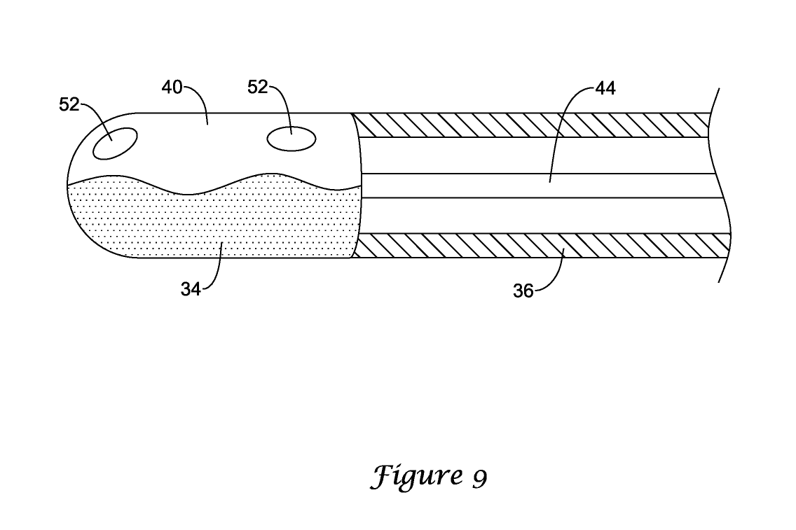

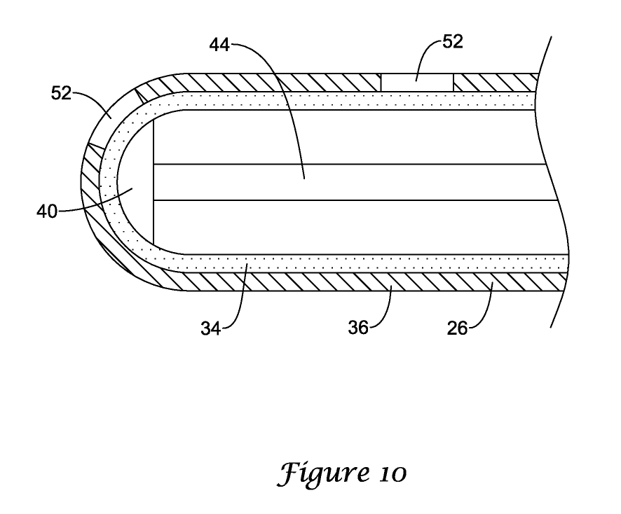

FIGS. 9 and 10 illustrate an embodiment where the wall 36 of the catheter surrounds the mesh 34. A conductor 44 extends to an electrode 40 disposed in the lumen of the catheter. One or more ports 52 are provided in the wall 36 of the distal end region to allow for the passage of fluid. A deflection wire (not illustrated) may be included.

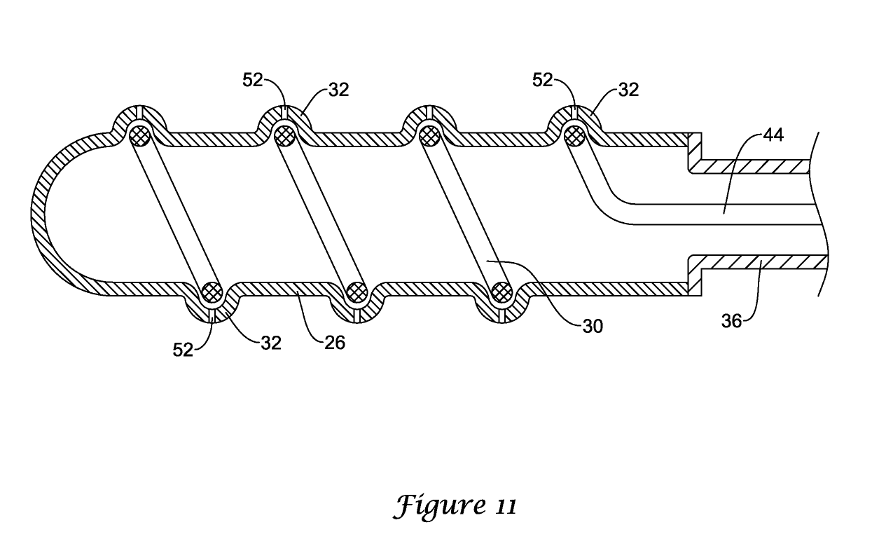

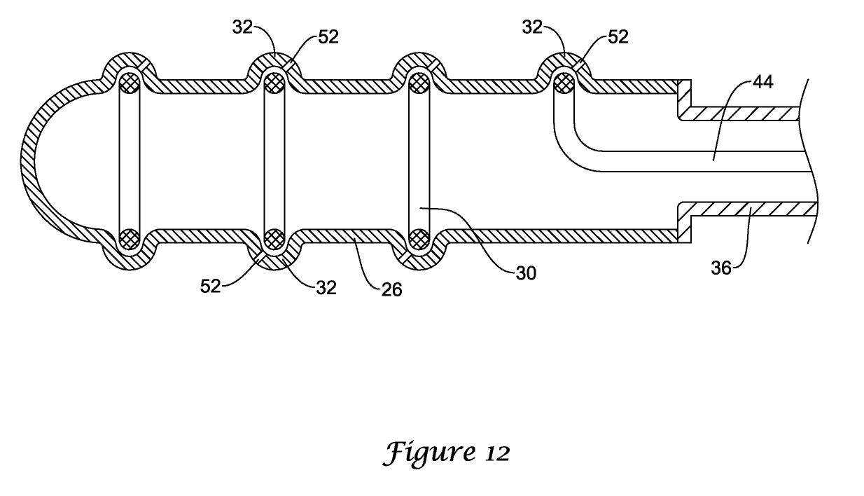

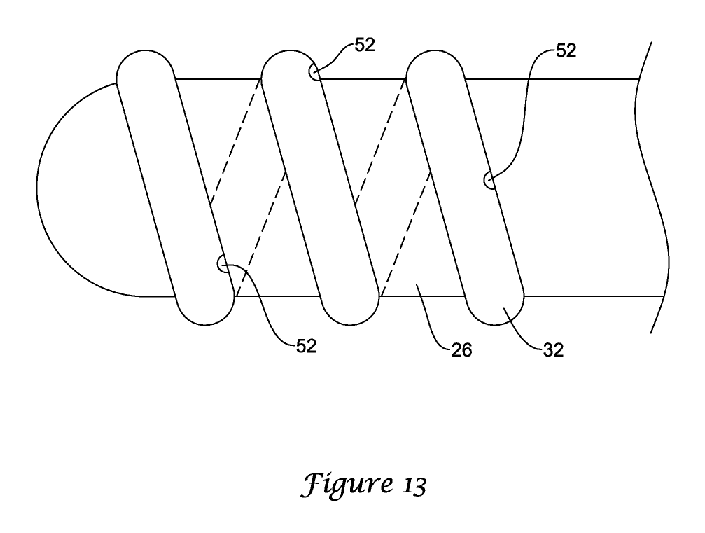

FIGS. 11-13 illustrate embodiments where the distal end region 26 of the catheter 12 includes spacers 32. An electrode such as coil 30 may be disposed within the distal end region. The spacers 32 may be hollow and may include a plurality of ports 52. A portion of the electrode may be disposed within the spacers. The ports may be face radially outwardly or may be directed generally proximally or generally distally. In some embodiments, some of the ports may be directed generally distally and some of the ports may be directed generally proximally. The ports may be located on side walls of the spacers such that the spacers are further radially out from the central longitudinal axis of the catheter than the ports. The spacers 32 may have a generally helical configuration, may comprise a plurality of annular rings. The spacers may comprise a plurality of discrete segments having gaps separating the discrete segments, as illustrated in FIG. 6. The embodiments may include a deflectable member such as a pull wire.

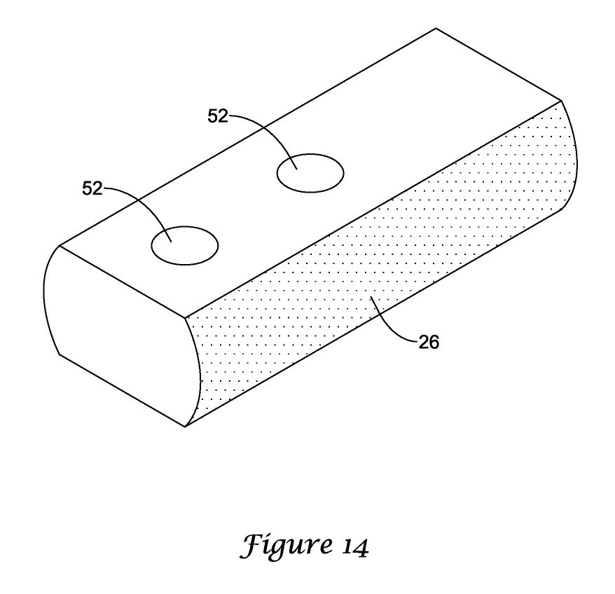

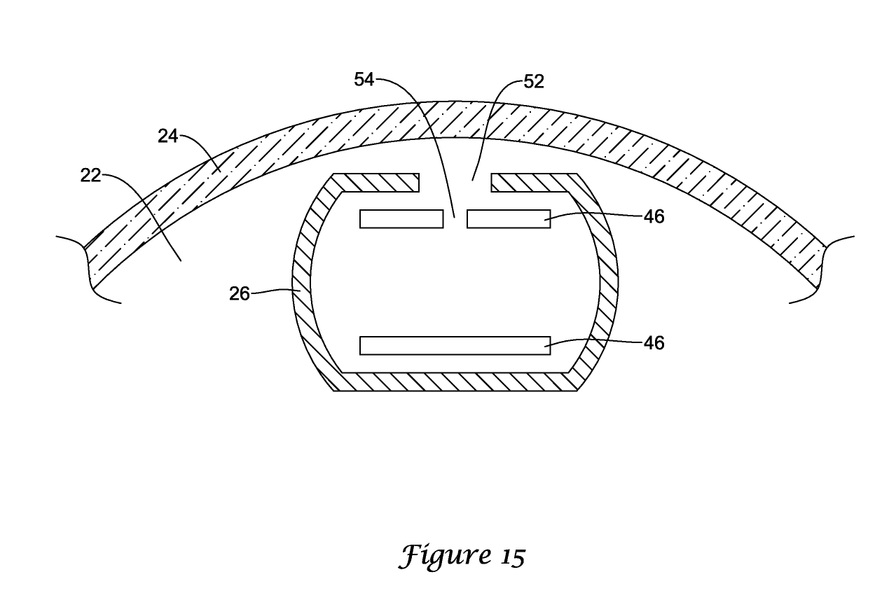

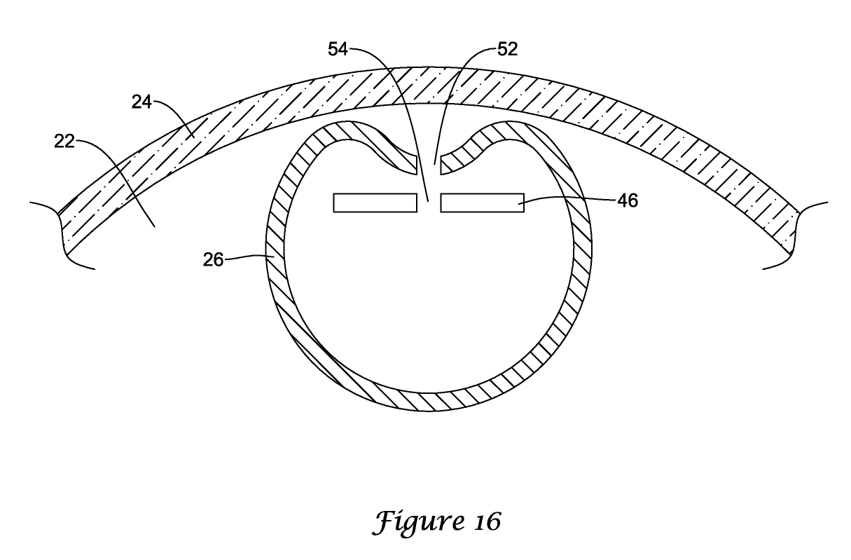

FIGS. 14 and 15 illustrate an embodiment where the distal end region 26 of the catheter has a non-circular cross-section. The cross-section may be oblong and may include one or more generally flat faces in the direction of the widest axis of the cross-section. One or more ports 52 may be disposed on the generally flat face. One or more ports may also be disposed on the distal end of the system. One or more conductors 46 may be located within the lumen of the catheter and may act as an electrode. The conductors 46 are generally flat ribbon shaped elements and may be located near a flat face. In the embodiment illustrated, a conductor 46 is located near a first flat face and a second conductor 46 is located near a second flat face. The conductors 46 may also act as deflection elements or pull wires. One or more lumen 54 may be provided in a conductor 46 to correspond with the one or more ports 52 in the flat face. FIG. 16 illustrates a variation that includes elongate ridges running along either side of the one or more ports 52.

The electrodes or conductors may be made of any suitable material such as copper, silver, gold, stainless steel, nickel, tin or a coated conductor or electrode such as a silver-coated stainless steel electrode.

In use, any of the systems described herein may be advanced through the vasculature in any manner known in the art. For example, system 10 may include a guidewire lumen to allow the system 10 to be advanced over a previously located guidewire. In some embodiments, the modulation system 10 may be advanced, or partially advanced, within a guide sheath such as the guide catheter 14 shown in FIG. 1. Once the distal end region 26 is placed adjacent to a desired treatment area, the guide catheter may be at least partially withdrawn to expose the distal end region. A deflection member may be actuated to position the distal end region near a treatment site. A conductive fluid such as a saline may be introduced through the lumen and the electrode may be activated to provide RF energy. The conductive fluid may be provided at a flow rate 2 cc/min to 10 cc/min or a flow rate of between 2 and 15 cc/mm or o between 10 and 30 cc/min or other desired flow rate. The RF energy is carried by the conductive fluid through the wall of the distal end region to treat the tissue of the vessel wall near the distal end region. Blood flow and conductive fluid flow at the treatment site may keep the intima of the vessel wall cool enough to prevent modulation of the intima. Nerve tissue in the media may be heated by the RF energy and denatured or ablated. Once a particular spot has been treated, the distal end region of the catheter may be moved to treat a second location. For example, the distal end region may be rotated and/or deflected to treat a second location on the same circumferential region of the vessel wall or may be rotated and withdrawn proximally to treat a second location on a different circumferential region of the vessel wall spaced longitudinally and circumferentially from the first treated location. This procedure may be repeated until a desired number of locations have been treated. In some instances, it will be desirable to treat a vessel wall such that the complete circumference of a vessel wall is treated. This circumferential coverage may be provided by treating regions that are spaced longitudinally from each other and are at different circumferential locations or may be provided by treating a complete circumferential ring of the vessel wall.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A system for nerve modulation, comprising
an elongate shaft having a proximal end region, a deflectable distal end region and a lumen extending to the deflectable distal end region; and
the deflectable distal end region comprising an electrode surrounded by a permeable membrane, the permeable membrane fluidly connected to the lumen.

2. The system of claim 1, wherein the elongate shaft further comprises a deflection wire extending from the proximal end region to the distal end region.

3. The system of claim 1, wherein the permeable membrane includes a woven mesh.

4. The system of claim 1, wherein the electrode includes a coil.

5. The system of claim 1, wherein the electrode comprises a flat ribbon.

6. The system of claim 1, wherein the electrode comprises a tube having a plurality of holes extending between an interior surface and an exterior surface.

7. The system of claim 1, wherein the deflectable distal end region further comprises a spacer that is disposed over the permeable membrane.

8. The system of claim 7, wherein the spacer is disposed radially around the deflectable distal end region.

9. The system of claim 7, wherein the spacer comprises a plurality of annular rings.

10. The system of claim 7, wherein the spacer comprises a helical member.

11. A system for nerve modulation, comprising
a catheter having an elongate shaft, a distal end region, a proximal end region and a lumen extending therebetween;
an electrode disposed in the distal end region; and
wherein the distal end region comprises one or more bumpers and a plurality of ports, the ports being fluidly connected to the lumen.

12. The system of claim 11, wherein the one or more bumpers are hollow and fluidly connected to the lumen.

13. The system of claim 11, wherein the one or more bumpers comprise a plurality of annular rings.

14. The system of claim 11, wherein the one or more bumpers comprise a helical member.

15. The system of claim 11, wherein the plurality of ports are disposed on the one or more bumpers.

16. The system of claim 11, wherein at least some of the plurality of ports are disposed on one or more side walls of the one or more bumpers.

17. The system of claim 11, wherein at least some of the plurality of ports face at least partially proximally.

18. The system of claim 11, wherein at least some of the plurality of ports face at least partially distally.

19. The system of claim 11, wherein the electrode includes a helical electrode.

20. A method of nerve modulation, the method comprising:
providing a medical device, comprising:
a catheter having an elongate shaft, a distal end region, a proximal end region and a lumen extending therebetween,
an electrode disposed in the distal end region, and
wherein the distal end region comprises one or more bumpers and a plurality of ports, the ports being fluidly connected to the lumen;
advancing the medical device through a blood vessel to a position adjacent to a renal artery; and
activating the electrode.
